(19) United States
(12) Patent Application Publication
(10) Pub. No.: US 2008/0208321 A1
(43) Pub. Date: Aug. 28, 2008

(54) POLYMERIC STENT AND METHOD OF MANUFACTURE

(75) Inventors: Subramanian VENKATRAMAN, Singapore (SG); Yin Chiang BOEY, Singapore (SG)

Correspondence Address: LEVINE BAGADE HAN LLP, 2483 EAST BAYSHORE ROAD, SUITE 100, PALO ALTO, CA, 94303, US

(73) Assignee: NANYANG TECHNOLOGICAL UNIVERSITY, Singapore (SG)

(21) Appl. No.: 12/109,317

(22) Filed: Apr. 24, 2008

Related U.S. Application Data
(62) Division of application No. 10/867,617, filed on Jun. 15, 2004.
(60) Provisional application No. 60/478,887, filed on Jun. 16, 2003.

(51) Int. Cl. A61F 2/06; B29C 47/06; B29C 47/00; B29C 65/00

(52) U.S. Cl. 623/1.19; 264/176.1; 156/60

(57) ABSTRACT

A stent formed of polymeric material, useful for the expansion of a lumen and the delivery of one or more therapeutic agents in situ is disclosed. The stent may be multi-layered, and may change shape at a state transition temperature governed by the materials forming the layers. Methods of use and manufacture are also disclosed.

### CROSS-REFERENCE TO RELATED APPLICATIONS

The present application is a divisional of U.S. patent application Ser. No. 10/867,617, filed on Jun. 15, 2004, which claims the benefit of U.S. Provisional Patent Application No. 60/478,887, filed Jun. 16, 2003, the contents of which are incorporated herein by reference in their entirety.

### BACKGROUND OF THE INVENTION

The present invention relates generally to medical devices for implanting in a patient, and particularly to stents that may be self expanding, and may deliver therapeutic agents.

Expandable medical prostheses, frequently referred to as stents, are well known and commercially available. They are, for example, disclosed generally in U.S. Pat. No. 4,655,771 (Wallsten), U.S. Pat. No. 5,061,275 (Wallsten et al.) and U.S. Pat. No. 5,645,559 (Hachtmann et al.). Stents are used within body vessels of humans for a variety of medical applications. Examples include intravascular stents for treating stenoses, stents for maintaining openings in the urinary, biliary, tracheobronchial, oesophageal and renal tracts and inferior vena cava.

Typically, a delivery device that retains the stent in its compressed state is used to deliver the stent to a treatment site through vessels in the body. Stents tend to be designed to be flexible with a reduced radius, to enable delivery through relatively small and curved vessels. In percutaneous transluminal angioplasty, an implantable endoprosthesis is introduced through a small percutaneous puncture site, airway or port and is passed through various body vessels to the treatment site. After the stent is positioned at the treatment site, the delivery device is actuated to release the stent and the stent is mechanically expanded, usually with the aid of an inflatable balloon, to thereby expand within the body vessel. The delivery device is then detached from the stent and removed from the patient. The stent remains in the vessel at the treatment site as an implant.

Commonly used materials for known stent filaments include Elgiloy™. and Phynoxim™. metal spring alloys. Other metallic materials that can be used for expandable stent filaments are 316 stainless steel, MP35N alloy and superelastic Nitinol nickel-titanium. Another expandable stent has a radiopaque clad composite structure such as shown in U.S. Pat. No. 5,630,840, naming Mayer. Expandable stents can also be made of a titanium alloy.

The implantation of an intraluminal stent may cause a certain amount of acute and chronic trauma to the luminal wall while performing its function. A stent that applies a gentle radial force against the wall and that is compliant and flexible with lumen movement is preferred for use in diseased, weakened or brittle lumens. Stents are preferably capable of withstanding radially occlusive pressure from tumours, plaque and luminal recoil and remodelling.

Certain stent designs tend to be self-expanding upon insertion within a lumen. For example, EP 1287790 (Schmitt & Lentz) describes an axially flexible braided stent that is self-expandable due to the elastic memory of the braided polymer fibres. The braided fibres are shaped into a tube at or just below the melting temperature of the polymer, and then longitudinally stretched upon cooling. The stent is inserted while stretched, and once inserted the stretch tension is released, allowing for the radial expansion of the tube when inserted.

Known self expanding stents, however, typically must be constrained to be inserted. Moreover, their removal is often difficult, if not impossible.

Accordingly, there is a need for improved expandable medical stents, that simplify insertion, and may simplify removal.

### BRIEF SUMMARY OF THE INVENTION

A polymer that is amorphous, or is at least partially amorphous, will undergo a transition from a pliable, elastic state (at higher temperatures) to a brittle glass-like state (at lower temperatures) as it transitions through a particular temperature, referred to as the glass transition temperature (T_{g}). The glass transition temperature for a given polymer will vary, depending on the size and flexibility of side-chains) as well as the flexibility of the backbone linkages and the size of functional groups incorporated into the polymer backbone. Below T_{g}, the polymer will maintain some flexibility, and may be deformed to a new shape. However, the further the temperature below T_{g} the polymer is when being deformed, the greater the force needed to shape it.

Furthermore, amorphous or partially amorphous polymers, when set into a particular shape at a higher temperature, have an elastic memory or shape memory, such that when cooled and compressed into a smaller shape, the polymer will expand back to the original shape upon heating above a state transition temperature. The terms “shape memory”, “elastic memory” and “memory effect” as used herein in respect of a polymer are interchangeable and refer to the characteristic of a polymer with a T_{g} to revert from one shape held below the T_{g} to a second shape when heated above the T_{g}, where the polymer has been previously set to the second shape above T_{g}.

This characteristic of amorphous or semi-crystalline polymers is employed in the self-expanding stent of the present invention. The present invention therefore provides, in one aspect, a stent. The term stent, as used herein, is intended to refer generally to expandable medical prostheses, including lengthwise extending stents, stent-grafts, grafts, filters, occlusive devices, valves or the like. The stent may be any suitable shape required to achieve the desired function as a medical prosthesis. For example, the stent may be generally tubular or generally helical.

As exemplified, the stent may be an implantable, helically tubular member which is an axially flexible and radially self-expandable structure comprising at least one polymeric layer. The stent assumes a substantially tubular form in the expanded or non-expanded state.

Such a stent may be useful for delivering therapeutic agents and, even more particularly, multiple therapeutic agents with multiple diffusion rates. The stent may be biostable or bioabsorbable.

The invention therefore provides in one aspect a stent comprising a substrate including a polymer that is at least partially amorphous and has a glass transition temperature T_{g}, and a therapeutic agent included in the polymer. The stent is formed to have a first shape at a lower temperature T₂ and a second shape at a higher temperature T₁ and configured to change from the first shape to the second shape at a temperature equal to or greater than a transition temperature T₃.

Exemplary stents may be formed having multiple layers. The layers may be arranged sequentially, relative to the helical width, thereby forming an outer and one or more inner layers. In an embodiment, a multiple layered stent has an outer layer formed from an amorphous polymer with a glass transition temperature (T_{g}) less than the T_{g} of a polymer that forms at least one inner layer.

Thus, in one aspect, the present invention provides a stent including at least first and second layers. The first layer includes a first polymer that is at least partially amorphous and has a glass transition temperature T_{g1}. The second layer includes a second polymer that is at least partially amorphous and has a glass transition temperature T_{g2}. The stent is formed to have a first shape at a lower temperature T₂ and a second shape at a higher temperature T₁, and configured to change from the first shape to the second shape at a temperature equal to or greater than a transition temperature T₃, dependent at least in part on at least one of T_{g1} and T_{g2}.

In another aspect, there is provided a stent including at least first and second layers. The first layer includes a first polymer and a first therapeutic agent. The second layer includes a second polymer and a second therapeutic agent. The stent is formed to have a first shape at a lower temperature T₂ and a second shape at a higher temperature T₁.

The incorporation of one or more polymer layers into the stent may offer several advantages: the self-expansion rate can be controlled through selection of appropriate polymers; the capability of delivering the same drug at two or more different rates is provided by using polymers that degrade at different rates; the capability of delivering two or more different drugs is also provided, for example by incorporating the different drugs into the different layers; and when drugs are to be incorporated, manufacturing processes may easily be employed which do not degrade the drugs. The present invention also contemplates methods of manufacturing the stents. In one aspect, the present invention provides a method of manufacturing a stent comprising forming a strip of polymer film having a first layer including a polymer that is at least partially amorphous and has a glass transition temperature T_{g1} and a second layer including a polymer that is at least partially amorphous and has a glass transition temperature T_{g2}; and shaping the strip into a first shape at a temperature T₁, wherein T₁=T_{g1}+X° C., and X is from about −20 to about +120. Additionally, the method may further comprise at a temperature T₂, shaping the strip into a second shape, wherein T₂=T₁−Y° C., and Y is from about 5 to about 80.

In another aspect, the invention provides a method of manufacturing a stent including: adding a therapeutic agent to a polymer that is at least partially amorphous and has a glass transition temperature; forming a strip of polymer film from the polymer; shaping the strip into a first shape at a temperature, wherein T₁=T_{g}+X° C., T_{g} is the glass transition temperature of the polymer and X is from about −20 to about +120; and at a temperature T₂, shaping the strip into a second shape, T₂=T₁−Y° C., and Y is from about 5 to about 80.

Such stents may be useful in a variety of medical applications where a body lumen, hollow organ or other cavity is desired to be de-constricted or de-restricted. Thus, such a stent is useful inter alia in the treatment of blockages or potential blockages and/or the prevention of restenosis of vascular, urinary, biliary, tracheobronchial, oesophageal and renal tracts. In an embodiment, the helical shape of the stent facilitates insertion of the stent and maintenance of the lumen in an open state.

Therefore, the invention provides in a further aspect a method of treatment or prophylaxis, to a subject in need of expansion of a lumen, comprising: introducing into the subject at site in the lumen desired to be expanded a stent comprising a first layer including a first polymer that is at least partially amorphous and a first therapeutic agent, thereby delivering the first therapeutic agent to the subject, the stent formed to have a first shape at a lower temperature T₂ and a second shape at a higher temperature T₁; and causing the stent to change to the second shape.

In a further aspect, the invention provides a method for prophylaxis or treatment of a subject in need of expansion of a lumen, comprising introducing into the subject at site in the lumen desired to be expanded a stent comprising a first layer including a first polymer that is at least partially amorphous and has a glass transition temperature T_{g1} and a second layer including a second polymer that is at least partially amorphous and has a glass transition temperature T_{g2}, the stent formed to have a first shape at a lower temperature T₂ and a second shape at a higher temperature T₁ and configured to change from the first shape to the second shape at a temperature equal to or greater than a shape transition temperature T₃, and wherein the introducing is performed at a temperature below T₃ such that the stent is in the first shape; and causing the stent to change to the second shape, in part by allowing the stent to equilibrate to a temperature equal to or greater than T₃.

Other aspects and features of the present invention will become apparent to those of ordinary skill in the art upon review of the following description of specific embodiments of the invention in conjunction with the accompanying figures.

### BRIEF DESCRIPTION OF THE SEVERAL VIEWS OF THE DRAWINGS

In the figures, which illustrate, by way of example only, embodiments of the present invention,

FIG. 1 is a side view of an stent, exemplary of an embodiment of the present present invention in a first state, having helical width D₁;

FIG. 2 is an end view of FIG. 1;

FIG. 3 is a side view of the stent of FIG. 1, in a second state, having helical width D₂;

FIG. 4 is an end view of FIG. 2;

FIG. 5 is a process flow diagram illustrating a method of manufacturing a stent, exemplary of an embodiment of the present invention;

FIG. 6 is a side view of a stent, exemplary of another embodiment of the present invention in a first state having helical width D₁;

FIG. 7 is an end view of FIG. 6;

FIG. 8 is a side view of the stent of FIG. 6, in a state having helical width D₂;

FIG. 9 is an end view of FIG. 8;

FIG. 10 is a side view of a stent formed of two side-by-side layers;

FIG. 11 is a flow diagram of a method of prophylaxis or treatment of a patient by introducing a stent into a lumen of the patient;

FIG. 12 is a graph of the self-expansion rates of particular single-layer and double-layer stents 37° C., with a target helical width of 3 mm; and

FIG. 13 is a representation of a catheter device comprising a balloon mechanism to deploy the helical medical stent.

### DETAILED DESCRIPTION OF THE INVENTION

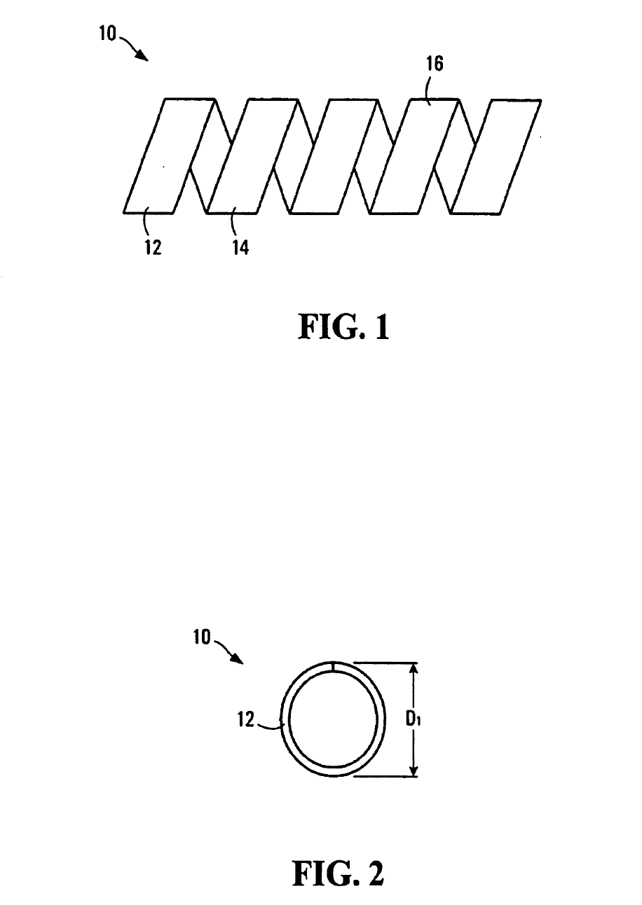

FIGS. 1-4, illustrate a stent 10, exemplary of one embodiment of the present invention. As illustrated, stent 10 includes a substrate 12 formed at least in part, from an amorphous polymer 14.

As will be appreciated, at the molecular level, amorphous polymers have at least a portion of the polymeric chains in a disordered state. Molecules are randomly arranged, having no long range order, rather than periodically arranged as in a crystalline material. As will be understood, such polymers therefore include polymers that are fully amorphous, partially amorphous and semi-crystalline. Amorphous polymers have a glass transition temperature T_{g} above which the polymer will be flexible as the polymer chains will be able to move relative to each other, and below which the polymer will be relatively brittle, since the polymer chains will tend not to move much relative to each other when the polymer is stressed. That is, below T_{g}, the material is solid, yet has no long-range molecular order and so is non-crystalline. In other words, the material is an amorphous solid, or a glass. Although brittle, the polymer may still be formed into another shape. The amount of force required to shape the polymer will increase the further the temperature at which the shaping is performed is below T_{g}. The glass transition temperature T_{g} is different for each polymer.

Generally, any polymer having a T_{g} may be used to form stent 10. Example polymers that may be used to form stent 10 include poly-L-lactide (PLLA), poly-D-lactide (PDLA), polyglycolide (PGA), poly(lactide-co-glycolide), polydioxanone, polycaprolactone, polygluconate, polylactic acid-polyethylene oxide copolymers, modified cellulose, collagen, poly(hydroxybutyrate), polyanhydride, polyphosphoester, poly(amino acids) or related copolymers material, polyurethane including physically cross-linked ether or ester-urethanes, polyethylene, poly(ethylene terephthalate) (PET), or Nylon 6,6.

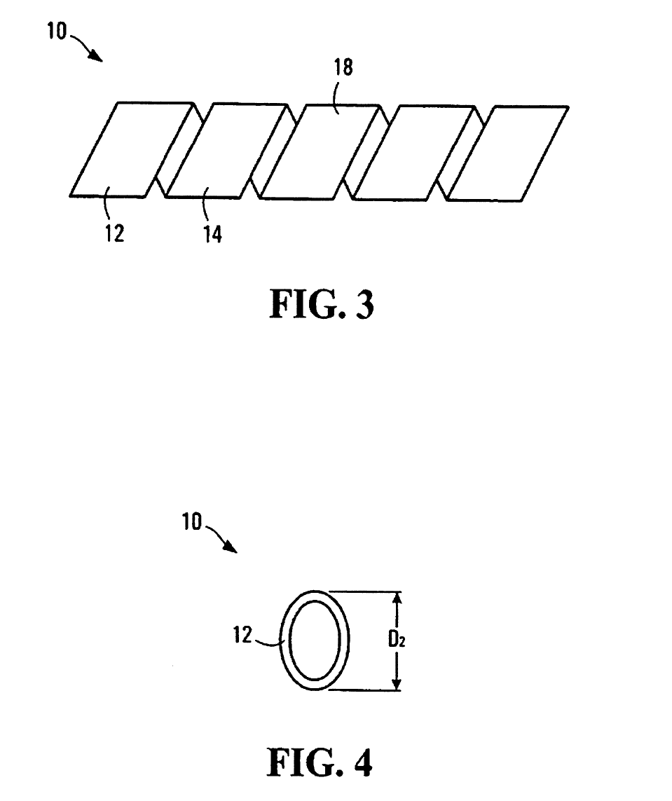

At a temperature below T_{g}, stent 10 is formed into its first state: a generally helical tubular shape 16 of helical width D₂ illustrated in FIGS. 3 and 4. At a second temperature above T_{g}, stent 10 is formed into its second state: a second generally helical tubular shape 18, having a helical width D₁ illustrated in FIGS. 1 and 2. In the depicted embodiment, shape 16 has a generally circular cross-section. As such, the helical widths D₁ and D₂ equal the helical diameters of the two helical shapes 16 and 18. Moreover, D₁/D₂>1. Thus, stent 10 is capable of self-expansion from its first state to its second at a given temperature, referred to as its state-transition temperature.

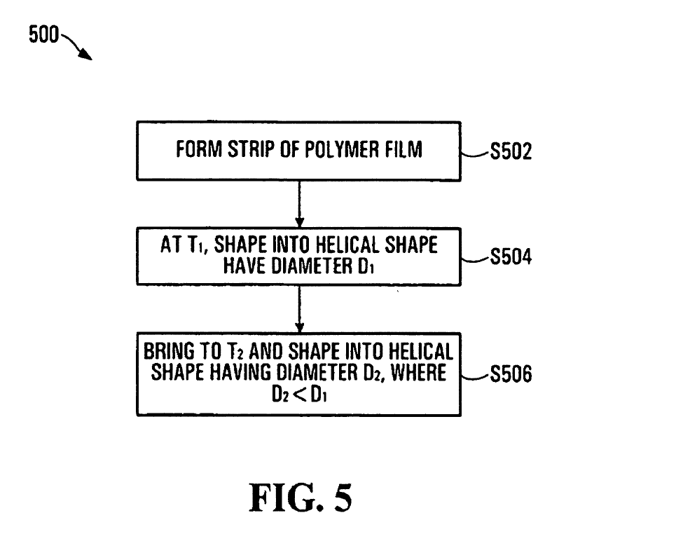

Stent 10 may be formed in accordance with method S500 illustrated in FIG. 5. As illustrated, in step S502, the substrate 12 is initially formed as a strip of polymer film.

The polymer film may be formed of one or more polymers, and may be formed using conventional methods known in the art, including solvent casting or extrusion of a polymer.

For example, a polymer to be extruded may be brought to an elevated temperature above its melting point. PLLA, for instance, may be heated to between 2.10° and 230° C. The polymer is then extruded at the elevated temperature into a continuous generally flat film using a suitable die, at a rate of about three to four feet per minute. The continuous film may then be cooled to or below T1, for example, by passing the film through a nucleation bath of water. The film is cut into a strip of desired width, if necessary.

In step S504 the film is brought to a temperature and set into a helical shape having helical diameter D₁. Typically, an oven is used to heat the film. T₁ is chosen somewhere above T_{g} for the polymer (i.e. T₁=T_{g}+X° C.). The value of X is from about −20 to about +120, typically from about 0 to about 30 or from about 0 to about 20. For PLLA, the oven temperature may be between about 60° C. and about 90° C. (preferably 70° C.).

The film is maintained at temperature T₁ for a period of time necessary to set the shape having diameter D₁. The period of time required to set D₁ will vary depending on T₁, T_{g} and the film thickness, and may be between 30 minutes and 24 hours.

Once set at the higher temperature of T₁, in S506 the stent is cooled to a lower temperature T₂, typically below T_{g} (i.e. T₂=T₁−Y° C.). At this temperature, the polymer may still be deformed, and is shaped into a helix of smaller helical width D₂, wherein D₂<D₁. This reduction in diameter is usually accompanied by an increase in length, as the helical stent is stretched. The value of Y is from about 5 to about 80, and typically from about 5 to about 50, more preferably from about 5 to 30. Typically, T₂, although below T_{g}, is close to T_{g}, for example, 5 to 20° C. below T_{g}. Usually, the closer T₂ is to T_{g}, the more easily the polymer can be shaped to D₂. At this smaller helical width, stent 10 is ready for use.

Finally, the film is collected onto spools of desired lengths.

Stent 10 so formed thus has two states: one having a helical shape of diameter D₂ (FIGS. 3 and 4); the other having a helical shape of diameter D₁ (FIGS. 1 and 2). As well, stent 10 will transition from its first state to its second state at or around a state transition temperature T₃. T₃ is a preferred temperature at which the stent 10 will expand, although the stent may expand above or below this temperature depending on how close T₃ is to T_{g}. Notably T₁<T₃<T₂. T₃ is related to the glass transition temperature of the polymer used to form stent 10. T₃ may be expressed as T₃=T_{g}+Z, where Z=−30 to +30. In the embodiment depicted in FIGS. 1-4, stent 10 is formed of a uniform film, made of the same polymer. In this instance, T₃ is about equal to T_{g}.

T₃ depends on the selected polymer and/or any additives. Preferably, it is a biologically relevant temperature. T₃ may, for example, be body temperature or below. Alternatively, the polymer may be chosen with T_{g}<37° C., T₃ may be equal to T₁. If T₃<37° C., prior to use special storage conditions may be required, such as storage at sub-ambient temperatures (or at least equal to or lower than T₂) or storage in a constrained state.

Optionally, a therapeutic agent may be incorporated into a stent so formed. The therapeutic agent may be included with the polymer prior to extrusion. Extrusion of the film allows inclusion of a drug or agent that can withstand the extrusion temperatures. The therapeutic agent may be any agent designed to have a therapeutic or preventative effect. For example, the therapeutic agent may be a drug, an antibiotic, an anti-inflammatory agent, an anti-clotting factor, a hormone, a nucleic acid, a peptide, a cellular factor, or a ligand for a cell surface receptor. As well, the therapeutic agent should be one that does not materially interfere with the physical or chemical properties of the polymer in which it is included.

Particular contemplated therapeutic agents include anti-proliferative agents such as sirolimus and its derivatives including everolimus, and paclitaxel and its derivatives; antithrombotic agents such as heparin, antimicrobial such as amoxicillin, chemotherapeutic agents such a spaclitaxel or doxorubicin, anti-viral agents such as ganciclovir, anti-hypertensive agents such as diuretics or verapramil or clonidine, and statins such as simvastatin.

Preferably, solvent casting, including spin casting, may be used to form film 16, since such casting does not use high temperatures, which may degrade many therapeutic agents. Such casting may facilitate incorporate numerous additional therapeutic agents. Thus, when a therapeutic agent is to be incorporated, solvent casting is preferred to extrusion and co-extrusion, as most therapeutic agents may degrade at extrusion temperatures.

Optionally, in order to reduce T_{g} a plasticizer may be added to the polymer prior to forming it into a film. Generally, a plasticizer is any solid or high-boiling liquid that is miscible with the polymer in the proportions used, and when the plasticizer has a T_{g}, referred to as T_{gp}, then T_{gp} is lower than T_{g} of the polymer. Acceptable plasticizers include low molecular weight liquids or solids, for example, glycerol, polyethylene glycol, carbon disulfide or triethyl citrate.

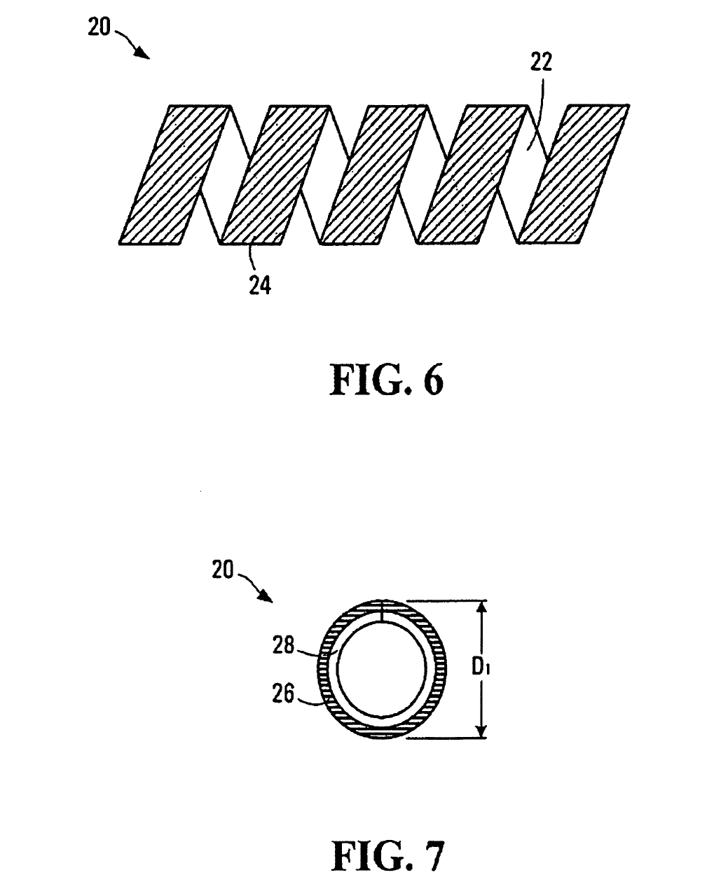

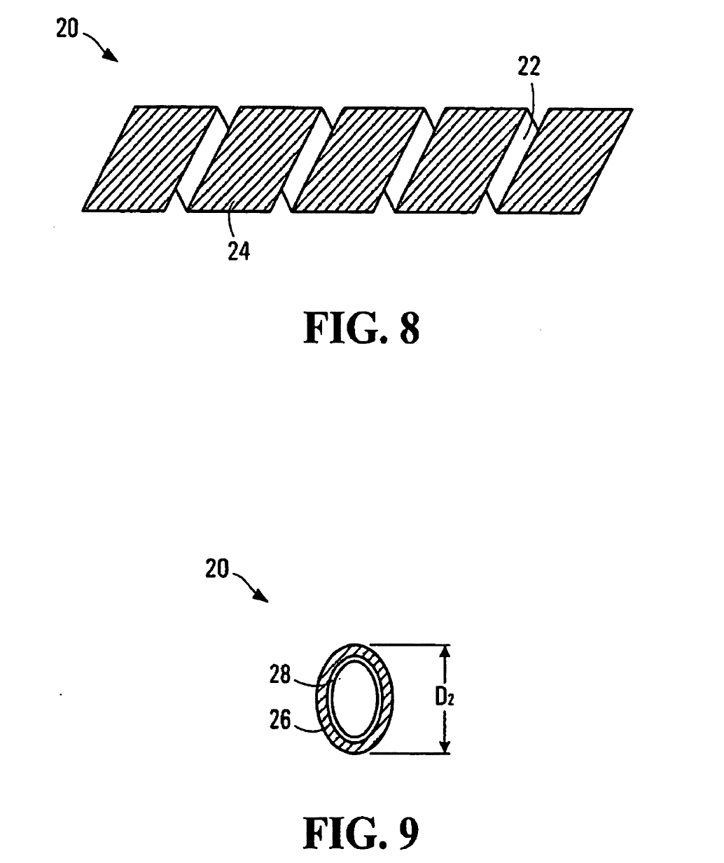

In a second embodiment, a stent 20 may be formed of one or more polymer layers 22, 24 as illustrated in FIGS. 6-9. As illustrated, layers 22 and 24 may be formed atop each other.

Layer 22 is arranged as the inner layer (closer to the axis of the helix) while layer 24 is the outer layer of the formed helix. The polymers forming the multiple layers have differing glass transition temperatures T_{g}. Outer layer 24, is formed of a first polymer 28, having a glass transition temperature T_{g1}; inner layer 22 is formed of a second polymer 26 having a different glass transition temperature T_{g2}. In the depicted embodiment, T_{g2} of the inner layer >T_{g}, of the outer layer. For example, the stent may be formed with an outermost layer having T_{g1} of between about 25° C. and 60° C., and an inner layer having a T₂ of between 60° C. and 100° C.

The outer layer, when above its T_{g}, pulls the inner layer, which may be below it T_{g2}, towards an expanded state, with the inner layer acting to dampen the expansion of the stent, influencing T₃, and the rate of expansion.

Again, suitable polymers from which the layer or layers 22, 24 of stent 20 may be formed include amorphous, partially amorphous and semi-crystalline polymers. The polymer may also be a cross-linked polymer such as generated via radiation, chemical process or physical pressure or manipulation.

Stent 20 may be formed in much the same way as stent 10 (FIGS. 1-4), as illustrated in FIG. 5. However, instead of extruding a single polymer to form a film, multiple layers may be co-extruded in step S502, thereby forming a multi-layer film. Interfacial bonding agents may be used to increase interlayer adhesion. For example, a solid surfactant such as Poloxamer® may be added to increase interfacial adhesion. For example, the surfactant may be added prior to extrusion. The resulting film will thus have two or more polymeric layers, atop of each other.

Alternatively, each of the multiple layers may be solvent cast. Such casting results in good interfacial adhesion. The second layer is cast from a solvent that does not dissolve the already-cast layer. For example, polyurethane used to form a first layer may be dissolved in dimethylformamide, while PET used to form a second layer may be dissolved in chloroform. The second solution may be spread on the first layer once dry, and the solvent evaporated off. Again, a surfactant may be added to the polymer solutions prior to casting. The resulting multi-layers have a strong bond between the layers.

The layers may alternatively be spin-cast using a high-speed spinning machine. Such a machine spins a solution of polymer onto a substrate and the solvent evaporates off. The films produced by this method may be thinner than those produced by solvent casting. This method can be used to produce multi-layered polymer films. Using this method, a very thin film, for example, having a total thickness of 0.1 to 0.2 mm, can be produced which contains up to 20 different polymer layers, with good interfacial bonding between adjacent layers.

A further alternative in making the polymer film is to extrude or cast an inner layer, then solvent-cast or spin-cast a cross-linkable layer onto the inner layer. Cross-linking may then be carried out by heat, pressure, or by the use of catalysts or by photo-initiation.

As with stent 10 described above, a suitable plasticizer may be added to one or more of the polymers prior to forming multi-layered stent 20, in order to reduce T_{g}, and where a plasticizer is added to more than one polymer, the same or different plasticizer may be added to each polymer.

In a preferred embodiment, a multi-layered helical stent is made by solvent casting an inner layer of PLLA in a solvent such as dichloromethane. The outer layer, such as PLGA, is made using a solvent such as acetone, which will not dissolve the PLLA. The solution is then cast onto the inner layer polymer and dried to generate a two-layered stent film. The film is then shaped helically as described above.

Once the multi-layered film is formed, it is again heated to T₁, and formed into a helical shape having helical diameter D₁. Thereafter it is cooled to T₂, and re-formed to a helical shape having diameter D₂. For multi-layer stent 20, the definitions of T₁ and T₂ are based on the T_{g1}, T_{g} of the outer polymer layer.

Conveniently T₃, the temperature at which a formed stent transitions from one state to another, is influenced by the T_{g}s of the multiple polymers (in the case of two layers T_{g1} of the first polymer 28 and T_{g2} of the second polymer 26). Typically, T₃ is closer to T_{g1}.

Similarly, the rate of expansion (i.e the rate at which stent 20 self-expands after its temperature has increased beyond the state transition temperature) may depend on the combination of polymers. For example, a single polymer generally has a slow expansion rate. For example, a poly-L-lactide (PLLA) of a medium molecular weight expands to its final helical width (D₁) at 37° C. in 300 hours (initial expansion of 135% occurs in 120 minutes). However, a medical device having two layers formed from, for example, PLLA and poly-lactoglycolide (PLGA), fully expands in 9 minutes at 37° C. The expansion rate may not be critical for many applications, such as for example, urological applications, in which a 24 to 48 hour expansion rate may be suitable. For other applications, such as for coronary artery applications, the expansion rate may be more critical. A skilled person will understand T₃ and the rate of expansion of the device by carefully selecting layers having particular T_{g}'s.

Generally, the rate of expansion is related to the difference between T₃ and T_{g}. The higher T₃ is above T_{g1}, the faster the expansion rate. The inclusion of an inner layer having T_{g2}>T_{g1} will influence the mechanical strength of the multi-layered stent 20 when in an expanded state, since the polymer of the outer layer may be above T_{g1}, and therefore lack the rigidity of the glass state. The inner layer, which may be below T_{g2}, and therefore still in a glass state, may therefore provide rigidity to the expanded stent.

Again, polymers suitable for use in one or more layers in the helical stent 20 include poly-L-lactide (PLLA), poly-D-lactide (PDLA), poly(lactide-co-glycolide), (PLGA), polyglycolide (PGA), polydioxanone, polycaprolactone, polygluconate, polylactic acid-polyethylene oxide copolymers, modified cellulose, collagen, poly(hydroxybutyrate), polyanhydride, polyphosphoester, poly(amino acids) or related copolymers material, polyurethane including physically cross-linked ether or ester-urethanes, polyethylene, poly(ethylene terephthalate) (PET), or Nylon 6,6.

In one embodiment, the medical device has at least two layers, For example, outer layer 24 may be formed from either an amorphous polymer with a T_{g} of between about 35° C. and about 60° C., or a cross-linked polymer with a T_{g} of between about 110° C. and about 60° C., and the second inner layer 22 is formed from either an amorphous or a semi-crystalline polymer with a T_{g} of between about 60° C. and about 1110° C., and where semi-crystalline, a crystalline melting point of greater than 100° C. In one example, the outer layer is made from PLGA 53/47, and the inner layer is made from PLA 8.4 or PLGA 80/20. For the aforementioned PLGA copolymers, the first number given in the polymer name refers to the PLA content (53% or 80%) while the second number refers to the PGA content (47% or 20%). It is also possible to use a plasticized PLA 8.4 (or other PLA) as the outer layer, such that its T_{g2} is between 40-60° C.

The use of cross-linked polymers, especially in the outer layer 24 is useful as the T_{g} of a cross-linked polymer may range from below body temperature to above body temperature, such as between about −10° C. and about 60° C. or more particularly between about 0° C. and about 40° C.

The relative thickness of the outer layer 24 and inner layer 22 can be varied, such that in different embodiments, the device, although having the same total thickness of the combined layers has a different thickness of the inner layer 22 and outer layer 24. For a two-layer stent, ratios of the inner layer 22 to outer layer 24 may be between 3:1 and 1:1.

In alternative embodiments, stent 20 may include additional layers formed from additional polymers. Again, the layers are preferably formed atop each other. The inclusion of multiple layers, each formed from a polymer having a different glass transition temperature, allows for a fine modulation of T₃, the state transition temperature of the device, as well as the rate at which the device expands to D₁. Where additional layers are included in stent 20, the T_{g} of each progressively more inward layer will be greater than the T_{g} of the previous more outward layer, such that the innermost layer will have the greatest T_{g}.

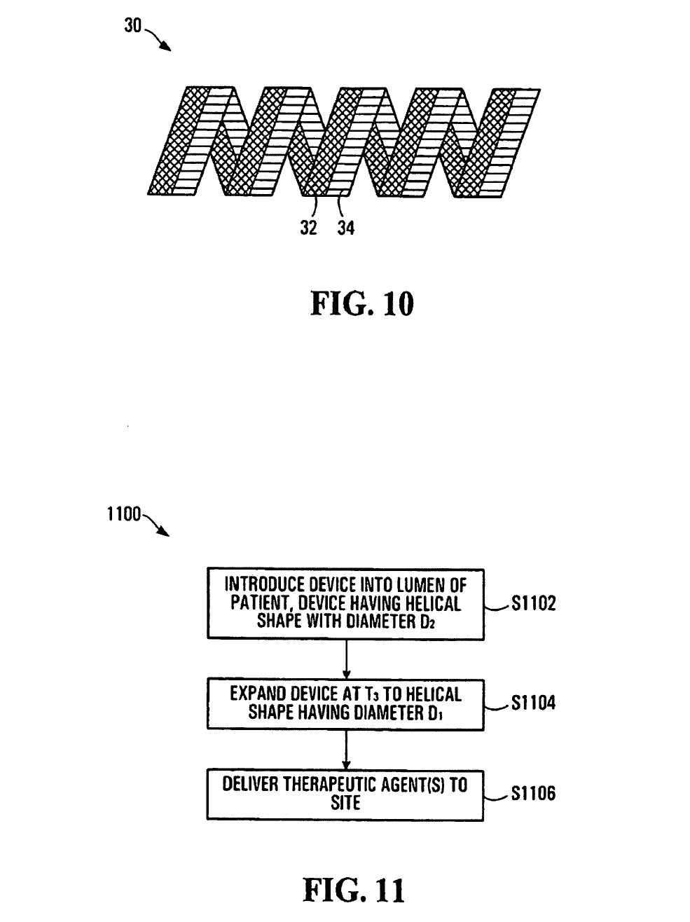

In yet further alternative embodiment, illustrated in FIG. 10, a two layered stent 30 may be formed with adjacent polymer layers rather than overlapping layers. As illustrated, the first layer 32 is positioned side-by-side relative to the second layer 34, such that the two layers wind the length of the helix, and such that the first layer 32 is above, being an upper layer, the second layer 32, being a lower layer, relative to the longitudinal axis of the helix. Again, stent 30 is formed with a general helical shape, having a helical diameter D₁, at temperature T₁. Thereafter, it is re-formed to a general helical shape having diameter D₂, at a temperature D₂.

Stent 30 is useful for the delivery of two or more therapeutic agents, or the delivery of a single therapeutic agent at differing rates. Therefore, stent 30 may include one or more therapeutic agents. For example, each layer may contain a different therapeutic agent, or each layer may contain the same therapeutic agent, which will be dispersed at different rates depending on the polymer used to form each layer and the different T_{g}s of the polymers. As the layers are formed side-by-side, the therapeutic agents will be delivered in the same direction.

Stent 30 is formed as described above, using co-extrusion or solvent-casting or spin-casting. The polymers used to form each layer may be co-extruded to form a polymer strip having adjacent bands of each polymer, such that when coiled into a helix the stent will have adjacent layers that wind the length of the helix. Alternatively, the layers may be cast side-by-side, typically with a small degree of overlap at the ends of the polymer strip.

For medical applications, the polymers used to form stent 10 (or stents 20, 30) are typically biocompatible, non-cytotoxic and non-allergenic, causing minimal irritation to the tissue when inserted in a lumen of a body.

In certain embodiments, the polymer or polymers used may be biostable, or non-biodegradable and are not degraded within the body. Such polymers are accepted to be substantially non-erodible in the sense that their erosion rates are usually of the order of years rather than months. Stent 10 (or stents 20, 30) formed of biostable polymers is particularly useful for applications for lumen de-restriction or de-constriction over long periods of time, as for example, in coronary artery applications or urological applications, or for use with cranial aneurysms. Suitable biostable polymers include polyurethanes, poly(ether urethanes), poly (ester urethanes), polycaprolactone, plasticized PVC, polyethylene, polyethylene terephthalate, polyvinyl acetate (PVAc), poly ethylene-co-vinyl acetate (PEVAc) or Nylon 6,6.

Stent 10 (or stents 20, 30), when constructed of a bioabsorbable polymer provides certain advantages over known devices such as metal stents, including natural decomposition into non-toxic chemical species over a period of time. A bioabsorbable device need not be retrieved using a second procedure after its useful life in the vessel. Also, bioabsorbable polymeric stents may be manufactured at relatively low costs since vacuum-heat treatment and chemical cleaning commonly used in metal stent manufacturing are not required. However, there may be certain situations where a biostable stent is the preferred option, for example in cardiovascular applications, for added safety beyond a 6-month period.

Stent 10 (or stents 20, 30) is designed to have good collapse strength (comparable to a metal stent), longitudinal flexibility (for ease of insertion) and easy expandability, so that it may be expanded inside the vessel or cavity, and then deployed by merely deflating the balloon. The self-expansion process is unique to the helical design. Stent mechanical properties and self-expansion are directly proportional to tensile modulus of the material. The invention advantageously provides polymeric stents with the required mechanical properties capable of bracing open endoluminal structures.

In an exemplary biostable two-layered stent 10 an outer layer 24 is made from polyurethane, which may be a physically cross-linked, for example a poly(ether urethane) or a poly(ester urethane), and an inner layer 22 made from poly(ethylene terephthalate) or Nylon 6,6.

Alternatively, one or more layers stent 20 (or stent 30) may be bioabsorbable. That is, various polymers degrade in the body but allow monomers and by-products to be absorbed. Bioabsorbable PLLA and PGA material, for example, degrade in vivo, through hydrolytic chain scission, to lactic acid and glycolic acid, respectively, which in turn is converted to CO₂ and then eliminated from the body by respiration.

Heterogenous degradation of semi-crystalline polymers, for example, typically occurs because such materials have amorphous and crystalline regions. Degradation occurs more rapidly at amorphous regions than at crystalline regions. This results in the product decreasing in strength faster than it decreases in mass. Totally amorphous, cross-linked polyesters show a more linear decrease in strength with mass over time as compared to a material with crystalline and amorphous regions. Degradation time may be affected by variations in chemical composition and polymer chain structures and material processing.

Suitable bioabsorbable polymers include poly-L-lactide (PLLA), poly-D-lactide (PDLA), polyglycolide (PGA), copolymers of lactide and glycolide (PLGA), polydioxanone, polygluconate, polylactic acid-polyethylene oxide copolymers, modified cellulose, collagen, poly(hydroxybutyrate), polyanhydride, polyphosphoester, poly(amino acids) or related copolymers, each of which have a characteristic degradation rate in the body. For example, PGA and polydioxanone are relatively fast-bioabsorbing materials (weeks to months) and PLLA and polycaprolactone are a relatively slow-bioabsorbing material (months to years). Thus, a skilled person will be able to choose an appropriate bioabsorbable material, with a degradation rate that is suitable for a desired application.

It should also be noted that the collapse pressures of two-layered stents are generally lower than with single layered stents, such as by a factor of half or more.

Generally, mechanical properties of polymers increase with increasing molecular weight. For instance, the strength and tensile modulus of PLLA generally increases with increasing molecular weight. PLLA, PDLA and PGA include tensile strengths of from about 40 thousands of pounds per square inch (ksi) (276 MPa) to about 120 ksi (827 MPa); a tensile strength of 80 ksi (552 MPa) is typical and a preferred tensile strength is from about 60 ksi (414 MPa) to about 120 ksi (827 MPa). Polydixanone, polycaprolactone and polygluconate include tensile strengths of from about 15 ksi (103 MPa) to about 60 ksi (414 MPa); a tensile strength of 35 ksi (241 MPa) is typical and a preferred tensile strength is from about 25 ksi (172 MPa) to about 45 ksi (310 MPa).

PLLA, PDLA and PGA include tensile modulus of from about 400,000 pounds per square inch (psi) (2,758 MPa) to about 2,000,000 psi (13,790 MPa); a tensile modulus of 900,000 psi (6,2606 MPa) is typical and a preferred tensile modulus is from about 700,000 psi (4,827 MPa) to about 1,200,000 psi (8,274 MPa). Polydioxanone, polycaprolactone and polygluconate include tensile modulus of from about 200,000 psi (1,379 MPa) to about 700,000 psi (4,827 MPa); a tensile modulus of 450,000 psi (3,103 MPa) is typical and a preferred tensile modulus is from about 350,000 psi (2,414 MPa) to about 550,00 psi (3,792 MPa).

A PLLA strip has a much lower tensile strength and tensile modulus than, for example, ELGILOY™. metal alloy wire which may be used to make braided stents. The tensile strength of PLLA is about 22% of the tensile strength of ELGILOY™. The tensile modulus of PLLA is about 3% of the tensile modulus of ELGILOY (registered trademark).

Stent 10 (or stents 20, 30) is generally radiolucent and the mechanical properties of the polymers are generally lower than structural metal alloys. Bioabsorbable or biostable stents may require radiopaque markers and may have a larger profile on a delivery catheter and in a body lumen to compensate for the lower material properties.

For example, an inner layer may be unplasticized, thereby having a high T_{g}, and an outer layer having a lower T_{g} may be produced by pre-plasticizing the same or a similar polymer with acceptable plasticizers. For example, a PLLA may be plasticized with glycerol, and cast or extruded on to a PGA layer. In this instance, the level of plasticization is so high as to render the PLLA amorphous, and making it more soluble in acceptable solvents.

In one embodiment, stent 20 is used to deliver a therapeutic agent in a biphasic pattern. Stent 20 is formed from two or more layers each having a different T_{g}, such that the same therapeutic agent may be dissolved or dispersed in the two or more layers, so as to diffuse out at different rates. The total amount of drug released may be manipulated by adjusting the thickness, T_{g} and the total area of the layer in which the drug is embedded. A skilled person, using routine experimentation, will be able to determine the appropriate amount of therapeutic agent to include in a particular layer in order to achieve a desired rate of release of the therapeutic agent, thereby delivering a particular dose of the therapeutic agent over time.

Conveniently, the innermost layer of stent 20 will release a therapeutic agent therein toward the longitudinal axis about which stent 20 winds. Similarly, the outermost layer of stent 20 will release a therapeutic agent therein away from the longitudinal axis about which stent 20 winds, and generally away from stent 20.

Where stent 20 (or 30) is formed of layers, if both layers are biodegradable, then the rate of biodegradation also influences the rate of drug release. In one embodiment, outer layer 24 is made from a first polymer 28 having a lower T_{g} and a faster degradation rate, and inner layer 22 is made from second polymer 26 having a higher T_{g} and a slower degradation rate. When in inserted into a lumen of a body, the outer layer 24 will generally degrade faster, leading to an initial fast rate of release of drug. Inner layer 22 will generally have a longer half-life, thereby remaining as substrate to keep the lumen open for the required length of time, while releasing drugs slowly over time.

Alternatively, a stent 20, exemplary of an embodiment of the present invention, allows for the delivery of two or more different therapeutic agents in a controlled fashion. In one embodiment, a multi-layered stent 20 having each layer formed from a polymer impregnated with one or more therapeutic agents, different from the therapeutic agent or agents included in other layers. The degradation rate and thickness of each layer may be designed such that the therapeutic agent or agents of each layer is released from the stent 20 at a particular rate or particular time period once inserted into the lumen.

For example, in the case of cardiovascular applications, a two-layered stent 20 is designed such that a non-proliferative drug is released initially at a faster rate from the outer layer 24, and then much more slowly from the inner layer 22 to prevent late-stage restenosis. In addition, the inner layer 22 may be used to deliver a different type of drug, such as an anti-coagulant, to the lumen side. There are other similar applications for a bi-phasic release profile for devices of the invention that will be understood by a person skilled in the art.

In use, stent 10 (or stents 20, 30) may be used in prophylaxis or treatment of a subject in need of expansion of a lumen, as illustrated in FIG. 11. Specifically, in step S1102, stent 10 is introduced into a lumen of a subject at a site that is desired to be expanded. The introduction is generally performed by inserting stent 10 at a temperature below T₃, while having helical width D₂. Stent 10 may be readily deployed in a lumen using a conventional catheter.

As will be appreciated, “lumen” as used herein refers to an inner open space or cavity of a tubular organ, including the cavity of a blood vessel, tubes of the gastrointestinal tract, ducts such as the bile duct, as well as the cavity of a ureter, the tube that leads from the kidney to the bladder.

In S1104, once at the desired location, stent 10 is expanded. This may be done by raising the temperature of the stent 10 to T₃. If T₃ has been chosen to be at or below body temperature, the device may self-expand as its temperature equilibrates to that of the implantation site.

However, although stent 10 is designed to self-expand, an additional expansion approach may be used, such as a biphasic expansion approach, for example, by a combination of radial expansion and raised temperature. If physical expansion is used, such expansion may be by balloon or bias-mediated expansion, as is known in the art.

After the deployment, and optionally expansion if by physical expansion, any deployment and expansion aids are removed. Conveniently, when the expansion is aided by a balloon, the balloon is deflated and removed. The prosthetic device is retained in place by the tissue with which it is in contact and its own expansion tendency.

Stent 10 may be partially expanded using a balloon and then left in place in the expanded state. Stent 10 may continue to expand to the defined final helical diameter D₁, and, if T₃ is designed to be equal to or less than 37° C., does not require heating to start the self-expansion process. This deployment of the helical stent will ensure that the blocked vessel or hollow organ is open and kept open for the duration of implantation, without complications arising from vessel or hollow organ recoiling.

Once deployed, stent 10 is generally shorter in length and larger in helical width than before deployment. For example, in one embodiment, the device may start out with a length of about 20 mm and helical width 1.5 mm and may reduce in length by about 15% and increase in helical width to about 3 mm after deployment. In comparison, an expandable metal stent generally has about the same longitudinal dimensions before loading and after deployment.

As will now be appreciated, stent 10 may be used in a variety of medical applications, including long-term and short-term implantation, where a biostable, rapidly degrading or slowly degrading bioabsorbable device is desired. Optionally, such stents may release one or more therapeutic agents at the implantation site. For example, stent 10 may be used in heart disease treatment, using bioabsorbable polymers with or without drug-carrying capacity, to prevent restenosis. Other applications include deployment of the present stents in thoracic surgery to keep airways open for patients suffering from bronchial stenosis, or in urology, to keep the ureter open.

Thus, in S1106, stent 10 (or stent 20, 30) delivers one or more therapeutic agent to the site of implantation where the device incorporates such therapeutic agents dispersed in one or more polymers used to form the device, as described above.

Typically, the diffusion of a drug through an amorphous or partially amorphous polymer is influenced by the T_{g} of the polymer; the diffusion rate of a drug is higher in polymers of lower T_{g}.

Of course, stents 10, 20 or 30 in the various embodiments as described above may be packaged for sale and sold with or without instructions for use.

Although the embodiments described herein relate to helical stents, a skilled person will appreciate that the invention is not so limited, and that the multi-layered polymeric stents and stents including therapeutic agents having the self-expansion properties described herein may be formed into shapes other than a helix, including a tubular shape.

Embodiments of the invention may be further appreciated, in light of the following non-limiting examples.

### EXAMPLES

### Example 1

### Manufacture of the Stent

A strip of polymer film is made by the usual methods (solvent-casting or extrusion). Next, the strip is coiled into a helical shape and set into this shape (helical width is D₁) higher temperature (T₁). The choice of T₁ depends on the T_{g} of the polymer: the general rule is to select T₁ such that T₁ is from T_{g} to about T_{g}+40° C. Once set at the higher temperature (T₁), the stent is usually made into a helix of smaller helical width (D₂); the ratio of D₁/D₂ is generally greater than 1, such as from 6 to 2) at a lower temperature (T₂): again, T₂ may range from T₁ less from about 5 to 80° C.

At this lower helical width, the stent may be deployed easily using a conventional catheter. Once inside the body vessel or cavity, the stent may be expanded by using both pressure and a raised temperature (this temperature is usually between T₁ and T₂ and is referred to as T₃, i.e. T₁>T₃>T₂). Under these conditions, the stent expands quickly first due to the physical expansion method and then more slowly due to the self-expansion properties of the stent, to the helical width set at T₁.

After the initial expansion, the balloon is deflated and withdrawn. The stent is retained in place by the tissue it is in contact with, and its own expansion tendency.

Generally, the stent, in use, is initially expanded by a balloon and then allowed to self-expand at body temperature. The expansion rate at body temperature is generally slower than at T₃, where T_{g} is below body temperature. FIGS. 1-4 provide a diagrammatic representation of the stent with helical widths D₁ and D₂.

### Example 2

### Generation of Multi-Layered Stent

The preferred configuration of the stent is a multi-layered helical stent, in which the outer layer(s) are made of an amorphous polymer with a T_{g} between 40° C. and 60° C., while the inner layer is made of an amorphous or semi-crystalline polymer with a higher T_{g} (60-100° C.), and crystalline melting point greater than 100° C. This ensures rapid expandability.

To make a two-layered stent, the following procedure is adopted. The inner layer (made from PLA, for example) is made by casting the polymer (with or without drug) from a solution in dichloromethane. A standard solution coater is used for this purpose. Next, a solution of the outer-layer polymer (typically a PLGA) is made in a solvent that does not dissolve the inner polymer that is already cast. An example of such a solvent is acetone. This solution is then cast onto the inner layer polymer, and dried to make the two-layer stent film. The film is then shaped into a helical stent using procedures already outlined above.

The two layers, if made from biodegradable polymers, will degrade at different rates, which may be used to advantage. For instance, in preventing restenosis, it appears that rapid neo-intimal cell proliferation occurs in the first 2-4 weeks. Thus the outer layer may be programmed to degrade over this period, releasing all the drug content in the same time period. The second layer may then be programmed to degrade at a much slower rate, to prevent later stage restenosis. It may also be used to deliver another drug, such as an anti-coagulant.

With a two (or multiple) layered system, the polymers may be on top of each other or side-by-side. The outer layer has a lower T_{g} than the inner layer or layers. In this case, the range of T₁ is usually from the T_{g} of the outer layer to about T_{g}+40° C. If the T_{g} of the outer polymer is close to 37° C., then the expansion rate is rapid at body temperature. In this instance, T₃ may be 37° C. Such is the case with PLGA 53/47, or a 50/50 copolymer of PLA and PGA, whose T_{g} is approximately 37-38° C.

Table 1 provides representative values for T₁, T₂ and T₃. Poly ethylene glycol was used as a plasticizer where indicated.

TABLE 1
T₁, T₁, and T₁ values

| POLYMER | T₁ | T₂ | T₃ |
|---|---|---|---|
| PLLA8.4 (T_{g} = 65° C.) | 50° C. | 25° C. | 37° C. |
| Single-layer | 70° C. | 40° C. | 45° C. (faster) or |
| | | | 37° C. |
| PLGA 80/20 (T_{g} = 51° C.) | 50° C. | 25° C. | 37° C. |
| Single-layer | 70° C. | 40° C. | 45° C. (faster) or |
| | | | 37° C. |
| PLLA8.4/plasticized PLGA | 37° C. | 25° C. | 37° C. |
| 80/20 (T_{g} = 44° C.) | | | |
| PLGA 80/20/plasticized | 50° C. | 25° C. | 37° C. |
| PLGA80/20/(T_{g} = 44° C.) | | | |

### Example 3

### Stent Expansion

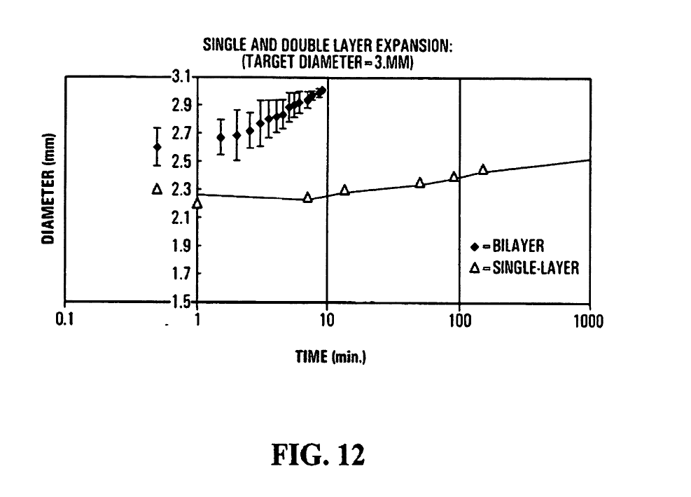

FIG. 12 is a graphical representation showing expansion rate data of for single-layer and double-layer stents at 37° C.

### Example 4

### Use of the Stent

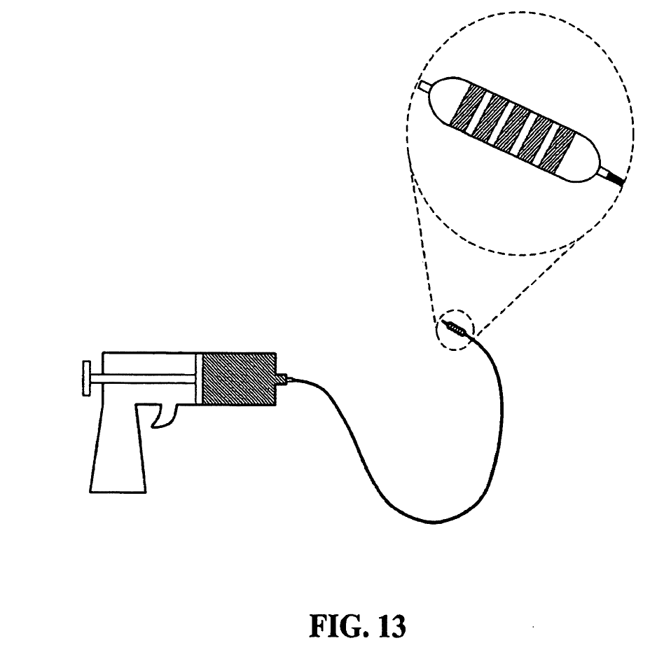

FIG. 13 is a representation of the stent being placed in situ.

### Example 5

### Therapeutic Agent Delivery

One or more polymers in the stent may be impregnated with a therapeutic agent or drug. Examples of such agents include anti-proliferative agents such as sirolimus and its derivatives including everolimus and paclitaxel and its derivatives; anti-thrombotic agents such as heparin; antibiotics such as amoxicillin; chemotherapeutic agents such a paclitaxel or doxorubicin; anti-viral agents such as ganciclovir; and anti-hypertensive agents such as diuretics or verapramil or clonidine.

While the helical shape herein described is preferred, it is possible to provide a fully tube-like stent which may be stretched to a lower helical width at a temperature greater than the T_{g} of any one of the polymers. This may require higher forces. The helical width may then be expanded at T₃ to provide a functional stent.

### Example 6

### Bilayer Stents

For a biostable PET/Poly vinyl acetate (PVA) stent, where T_{g} of PVA (outer layer)=28° C. and T_{g} of PET (inner layer)=+60° C., and where T₁=37° C. and T₂=25° C., a self-expanding stent having a PET layer with thickness=0.18 mm; PVA thickness=0.07 to 0.15 mm.

An extruded sheet of PET, 0.18 mm thick, is used as the inner layer. On to this is cast a PVA film, using a solution of PVA in dichloromethane. The thickness of the cast layer of PVA is about 0.10 mm. This bilayer film is set into a helical stent of helical width 3 mm at 37° C. for 1 hour and the lower helical width of 1 mm is set at 25° C. This stent can be balloon-expanded and then self-expand at 37° C.

As will now be appreciated, the above describe embodiments are susceptible to many modifications. For example, an exemplary stent could be formed of a non-helical shape. An exemplary stent could be formed of having a generally cylindrical shape, two differing shapes at two temperatures, or an undefined shape at one temperature. Similarly, exemplary stents could be formed of third, fourth and additional layers, between first and second layers. Each or some of the multiple layers could include a therapeutic agent as described.

As can be understood by one skilled in the art, many modifications to the exemplary embodiments described herein are possible. The invention, rather, is intended to encompass all such modification within its scope, as defined by the claims. The invention also includes all of the steps, features, compositions and compounds referred to or indicated in this specification, individually or collectively, and any and all combinations of any two or more of the steps or features.

We claim:

1. A method of forming an implantable prosthesis, comprising:
forming an extruded polymer that is formed into a structure having a first diameter at a first temperature;
radially altering the structure to have a second diameter which is different from the first diameter; and
cooling the structure to a second temperature lower than the first temperature such that the second diameter is set in the structure.

2. The method of claim 1 wherein forming comprises extruding a continuous film of polymer.

3. The method of claim 1 wherein forming further comprises cooling the polymer to a temperature at or below the first temperature.

4. The method of claim 1 wherein the first temperature, T₁, is determined whereby T₁=T_{g}+X° C., where T_{g} is a glass transition temperature of the polymer and X is from about −20 to about +120.

5. The method of claim 4 wherein the second temperature, T₂, is determined whereby T₂=T₁−Y° C., and Y is from about 5 to about 80.

6. The method of claim 1 wherein forming comprises forming the structure to have a helical shape.

7. The method of claim 1 wherein radially altering the structure comprises applying a force to the structure such that the diameter is altered from the first diameter to the second diameter.

8. The method of claim 1 wherein radially altering the structure comprises urging the structure from the first diameter, D₁, to the second diameter, D₂, wherein D₂<D₁.

9. The method of claim 1 further comprising mixing an agent which is miscible with the polymer prior to extruding.

10. The method of claim 1 further comprising adding a therapeutic agent to the polymer prior to extruding.

11. The method of claim 10 wherein said therapeutic agent is selected from the group consisting of a drug, an antibiotic, an anti-inflammatory agent, an anti-clotting factor, a hormone, a nucleic acid, a peptide, a cellular factor, a ligand for a cell surface receptor, an anti-proliferative agent, an antithrombotic agent, an antimicrobial agent, an anti-viral agent, a chemotherapeutic agent, and an anti-hypertensive agent.

12. The method of claim 1 wherein the polymer comprises a biostable polymer selected from the group consisting of polyethylene, polypropylene, polyethylene terephthalate (PET), polyurethane poly(ether urethane), poly(ester urethane), poly vinyl chloride, polyvinyl acetate (PVAc), poly(ethylene-co-vinyl acetate) (PEVAc), polycaprolactone and Nylon 6,6.

13. The method of claim 1 wherein the polymer comprises a bioabsorbable polymer selected from the group consisting of poly-L-lactide (PLLA), poly-D-lactide (PDLA), polyglycolide (PGA), polylactide-co-glycolide (PLGA), polydioxanone, polygluconate, polylactic acid-polyethylene oxide copolymer, modified cellulose, collagen, poly(hydroxybutyrate), polyanhydride, polyphosphoester and poly-amino acid.

14. A method of inducing stability into an implantable prosthesis, comprising:
forming a structure at a first temperature from an extruded polymer such that the structure defines a first diameter;
setting the first diameter in the structure;
reconfiguring the structure at a second temperature lower than the first temperature to define a second diameter different from the first diameter; and
setting the second diameter in the structure.

15. The method of claim 14 wherein forming comprises extruding a continuous polymer film.

16. The method of claim 14 wherein setting the first diameter comprises cooling the polymer to a temperature at or below the first temperature.

17. The method of claim 14 wherein reconfiguring comprises applying a force to the structure such that the diameter is altered from the first diameter to the second diameter.

18. The method of claim 14 wherein reconfiguring comprises urging the structure from the first diameter, D₁, to the second diameter, D₂, wherein D₂<D₁.

19. The method of claim 14 further comprising adding a therapeutic agent to the polymer prior to forming.

20. A method of forming an implantable prosthesis, comprising:
forming a co-extruded polymer having a first and a second layer, wherein the first layer comprises a first polymer having a glass transition temperature T_{g1} and wherein the second layer comprises a second polymer having a glass transition temperature T_{g2};
forming a structure having a first diameter at a first temperature;
radially altering the structure to have a second diameter which is different from the first diameter; and
cooling the structure to a second temperature lower than the first temperature such that the second diameter is set in the structure.

21. The method of claim 20 wherein co-extruding comprises extruding the first layer and second layer independently of one another.

22. The method of claim 21 further comprising attaching the first layer and second layer to one another via an attachment mechanism selected from the group consisting of thermal bonding, solvent bonding, adhesive bonding, mechanical friction, and combinations thereof.

23. The method of claim 20 wherein that the first layer forms an outer surface of the structure and the second layer forms an inner surface of the structure.

24. The method of claim 20 wherein radially altering comprises applying a force to the structure such that the diameter is altered from the first diameter to the second diameter.

25. An implantable prosthesis, comprising:
a substrate having a first polymer that is at least partially amorphous and has a glass transition temperature T_{g1}, and a second polymer that is at least partially amorphous and has a glass transition temperature T_{g2},
wherein the substrate is formed as a structure from a co-extruded polymer such that the structure has an inner surface and an exterior surface for contacting a vessel wall, and
wherein the structure is formed to have a first shape at a first temperature and a second shape at a second temperature different from the first temperature and configured to change from said first shape to said second shape at a temperature equal to or greater than a transition temperature.

26. The method of claim 25 wherein co-extruding comprises extruding the first layer and second layer independently of one another.

27. The method of claim 26 further comprising attaching the first layer and second layer to one another via an attachment mechanism selected from the group consisting of thermal bonding, solvent bonding, adhesive bonding, mechanical friction, and combinations thereof.

28. The prosthesis of claim 25 wherein the structure is formed from the first shape to the second shape when urged via application of a force.
